(19) United States
(12) Patent Application Publication
(10) Pub. No.: US 2014/0137873 A1
(43) Pub. Date: May 22, 2014

(54) Condom

(71) Applicant: Shawn Wright, St. Catherine (JM)

(72) Inventor: Shawn Wright, St. Catherine (JM)

(21) Appl. No.: 13/681,834

(22) Filed: Nov. 20, 2012

(51) Int. Cl. A61F 6/04

(52) U.S. Cl. CPC A61F 6/04
USPC 128/844

(57) ABSTRACT

An improved condom is disclosed. The condom consists of an elongated resilient and flexible tube having a closed end, an open end and a cylindrical wall extending between the open and closed ends. An elongated channel is formed in fee wail and extends between die closed end and the open end. The elongated channel has a first opening adjacent the closed end of the tube and a second opening adjacent the open end of the tube. The flexible tube, channel and openings being configured such that air can be forced loan the closed end, through the elongated channel and out the open end alter the condom is worn by progressively pressing against the condom in the vicinity of the channel from the closed end towards the open end.

### FIELD OF THE INVENTION

The invention relates generally to condoms.

### BACKGROUND OF THE INVENTION

Condoms are a common method of preventing sexually transmitted diseases and birth control. All condoms consist of an elongated tube having one closed end arid one open end. The closed end is often provided with a narrow and slightly elongated dimple, or accumulator tip, which serves to accumulate and store a quantity of semen, thereby lowering the likelihood that the semen will be accidental release. While these condom designs have been widely accepted, there is one principle problem with the accumulator up design of condoms. The accumulator portion of the condom has the tendency to retain air when the condom is worn. The air contained in the condom can cause the condom to fail in use. Therefore, to prevent condom failure, care must be taken to ensure that no air is trapped in the accumulator tin when the condom is worn, a task which is not always simple or easy to perform. An improved condom design which does not suffer from this drawback is therefore required.

### SUMMARY OF THE INVENTION

In accordance with one aspect of the present invention, there is provided a condom consisting of an elongated tube of flexible polymer having an open end, a closed end and a cylindrical wall. The closed end of the condom is provided with an elongated dimple which acts as an accumulator tip. An enclosed elongated channel is formed en the cylindrical wall of the condom and extends from the accumulator tip to the open end. The enclosed elongated channel having a first opening at the accumulator tip and a second opening at the open end of the condom.

With the foregoing in view, and other advantages as will become apparent to those skilled in the art to which this invention relates as this specification proceeds, the invention is herein described by reference to the accompanying drawings forming a part hereof, which includes a description of the preferred typical embodiment of the principles of the present invention.

### DESCRIPTION OF THE DRAWINGS

FIG. 1 is a top view of a condom made in accordance with the present invention showing the relative position of the enclosed channel.

FIG. 2 is a cross section view of the condom shown in FIG. 1 taken along line A-A.

FIG. 3 is a top view of an alternate embodiment of a condom made in accordance with the present invention showing a curved enclosed channel.

In the drawings like characters of reference indicate corresponding pasts in the different figures.

### DETAILED DESCRIPTION OF THE INVENTION

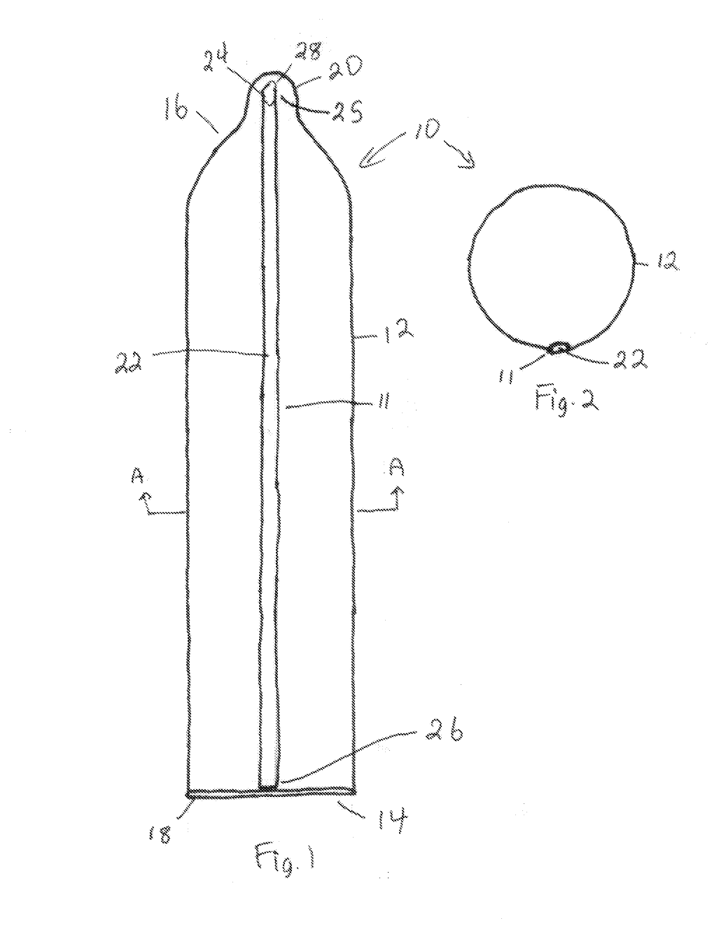

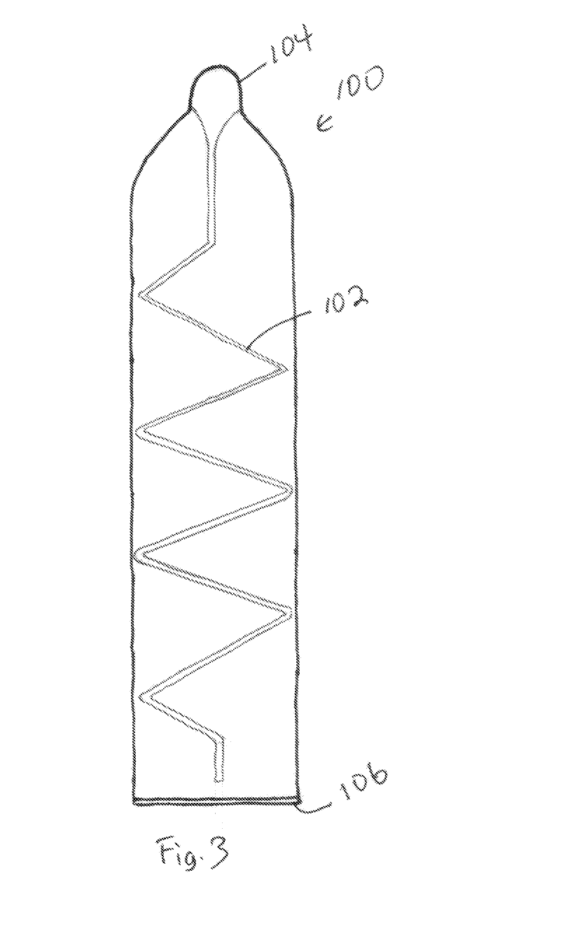

Referring to FIG. 1, a condom made in accordance with the present invention is shown generally as item 10 and includes an elongated tube shown generally as item 12 preferably made of a flexible and resilient polymer such as latex. Tube 12 has open end 14 and closed end 16. Bead 18 is formed on open end 14 to aid in applying the condom. An elongated dimple (accumulator tip) 20 is formed on closed end 16 at the very tip of the closed end. An elongated channel 22 is formed in elongated wall portion 11 of tube 12 and extends front open end 14 to dimple 20. Elongated channel 22 has openings positioned at end 24 positioned at accumulator tip 20 and end 26 positioned adjacent open end 18. Channel 22 is formed in elongated portion wall 11 of tube 12 which extends between ends 16 and 14 as is sufficiently thick so as to accommodate the channel. After condom 10 is applied, air can be vented from reservoir 20 to open end 14 by means of elongated channel 22. Opening 24 is configured to remain open even after the condom is worn. This can be achieved by making portion 25 of the condom adjacent opening 24 thicker, or by shaping opening 24 elliptically 28, as illustrated.

The polymer used to make condom 10 should be of sufficient residence and strength, and portion 11 of the wall of the condom is made sufficiently thick to permit air to be forced through channel 22 by applying pressure along the outside of the condom progressively from opening 24 to opening 26. When the condom is first worn, air will tend to accumulate in accumulator tip 20. Squeezing tip 20 will cause air to pass through opening 24 and into channel 22. The outside of the condom is then physically pressed or rubbed to drive the air through channel 22 and out opening 26 which then vets through open end 14.

The flexibility and thickness of the polymer used to construct condom 10 should be selected to ensure that elongated channel 22 collapses when the condom is applied. The collapsed channel can be opened by forcing air through the channel from the accumulator tip, as described above. Preferably, the thickness and stiffness of the polymer used to make the tube should not be so great as to cause the elongated channel to remain open after the condom is applied, because this may permit semen to pass through the channel thereby defeating the purpose of wearing the condom.

Referring now to FIG. 3, the elongated channel which extends from the accumulator tip to the open end need not be straight, but instead may be sinusoidal. Condom 100 is identical to the previous embodiment shown, the only difference being that channel 102 extends sinusoidally from tip 104 to open end 106. The sinusoidal pattern may make it more difficult for semen to accidentally pass through the channel and out the open end.

A specific embodiment of the present invention has been disclosed; however, several variations of the disclosed embodiment could be envisioned as within the scope of this invention. It is to be understood that the present invention is not limited to the embodiments described above, but encompasses any and all embodiments within the scope of the following claims

Therefore, what is claimed is:

1. A condom comprising an elongated resilient and flexible tube having a closed end, an open end and a cylindrical wall extending between the open and closed ends, an elongated channel formed in the wall extending between the closed end and the open end, the elongated channel having a first opening adjacent the closed end of the tube and a second opening adjacent the open end of the tube.

2. The condom of claim 1 wherein an accumulator reservoir is formed on the closed end of the flexible tube, the first opening positioned at the accumulator reservoir, the first opening configured, to permit air to vent there the accumulator reservoir through the elongated channel when the condom is worn.

3. The condom of claim 1 wherein the elongated channel is sinusoidal.

4. The condom of claim 2 wherein an elongated portion of the cylindrical wall extending between the accumulator reservoir and open end is sufficiently thick to accommodate the elongated channel and sufficiently resilient to permit air to be forced along the channel after the condom is worn by progressively pressing against the elongated portion of the cylindrical wail from the first opening towards the second opening.

5. The condom of claim 4 wherein the elongated portion of the cylindrical wall is sufficiently resilient to collapse the elongated channel when the condom is worn and there is no air in the elongated channel.
